(19) United States
(12) Patent Application Publication
(10) Pub. No.: US 2022/0137041 A1
(43) Pub. Date: May 5, 2022

(54) DEVICE FOR METHODS OF DETECTING CANCER

(71) Applicant: UNIVERSITY OF MIAMI, Miami, FL (US)

(72) Inventor: Elizabeth FRANZMANN, Miami, FL (US)

(21) Appl. No.: 17/433,815

(22) PCT Filed: Feb. 26, 2020
(86) PCT No.: PCT/US2020/019807

§ 371 Date: Aug. 25, 2021

Related U.S. Application Data
(60) Provisional application No. 62/810,551, filed on Feb. 26, 2019.

(51) Int. Cl. G01N 33/543; G01N 33/574; G01N 33/68; G01N 33/53

(52) U.S. Cl. CPC G01N 33/54388; G01N 33/57496; G01N 2333/70585; G01N 33/5302; G01N 33/6827

(57) ABSTRACT

A lateral flow assay device for testing a biological sample includes housing, a sample receiving pad, a conjugate test pad, and a nitrocellulose membrane. The sample receiving pad and conjugate test pad, as well as the nitrocellulose membrane, are enclosed within an interior portion of the housing. The sample receiving pad is in fluid communication with an opening defined in an outer surface of the housing for receiving the biological sample. At least a portion of the conjugate test pad is in contact with the sample receiving pad and is configured to test the biological sample. At least one window is defined in the outer surface of the housing adjacent the conjugate test pads such that the results of the test performed on the conjugate test pads are visible from outside of the housing.

### CROSS-REFERENCE TO RELATED APPLICATIONS

This application claims benefit of U.S. Provisional Application No. 62/810,551, filed Feb. 26, 2019, which is hereby incorporated by reference in its entirety.

### FIELD OF THE INVENTION

The disclosure relates generally to a device for detecting states of diseases, and more specifically related to a device having one or more strips for early detection of disease states.

### BACKGROUND OF THE DISCLOSURE

Head and neck squamous cell carcinoma (HNSCC) is a debilitating and deadly disease marked by disparities, with a mortality rate in blacks twice that of whites. It is more common in men than women (Cancer Facts and Figures-2013. Atlanta: American Cancer Society, 2013). HNSCC accounts for almost 90% of cancers involving the upper aerodigestive tract (UADT) (Muir et al. Upper aerodigestive tract cancers. Cancer Suppl (1995) 75:147-53). Each year the disease affects 50,000 people in the United States and 600,000 people world-wide. Survival rates are poor because most patients present in late stage when cure rates are as low as 30% (Vokes, et al. Head and neck cancer. N Engl J Med (1993) 328:184-94). The disease can be cured 80-90% of the time if detected early (Markopoulos, et al. Salivary Markers for Oral Cancer Detection. Open Dent J. (2010) 4:172-178).

Many of the current HNSCC biomarker studies use “omics” approaches to identify a few candidate markers from a large pool of potential markers using case-control study design (Shankar, et al. Trends in salivary diagnostics—a 5-year review of oral oncology 2007-2011. Oral Oncol (2012) 48:e22-3; Li, et al. Salivary transcriptome diagnostics for oral cancer detection. Clin Cancer Res (2004) 10:8442-8450; Park, et al. Salivary microRNA: discovery, characterization, and clinical utility for oral cancer detection, Clin Cancer Res (2009) 15:5473-5477; Hu, et al. Clin Cancer Res (2008) 14:6246-6252; Carvalho, et al. Evaluation of promoter hypermethylation detection in body fluids as a screening/diagnosis tool for head and neck squamous cell carcinoma. Clin Cancer Res (2008) 14:97-107; Elashoff, et al. Prevalidation of salivary biomarkers for oral cancer detection. Cancer Epidemiol Biomarkers Prev (2012) 21(4):664-72). On further testing, the markers usually fail to perform as well. For example, panels of methylation markers yielded sensitivity of 35-85% and specificity of 30-90% (Carvalho, et al. Evaluation of promoter hypermethylation detection in body fluids as a screening/diagnosis tool for head and neck squamous cell carcinoma. Clin Cancer Res (2008) 14:97-107) while a panel of mRNA markers yielded sensitivity of 45-79% and specificity of 72-77% (Elashoff D, et al. Prevalidation of salivary biomarkers for oral cancer detection. Cancer Epidemiol Biomarkers Prev (2012) 21(4):664-72).

The current “gold standard” for screening is physical examination followed by biopsy, but sensitivity is only 64% and specificity is 74% (Brocklehurst, et al. Screening programmes for the early detection and prevention of oral cancer. Cochrane Database Syst Rev (2010) 11:CD004150). A variety of technologies exist to aid in the detection of oral cancers, but there is no evidence that they are any better than the naked eye for screening. What is needed in the art is a point-of-care device for easily identifying a risk of cancer, detecting cancer, providing a prognosis of cancer, or monitoring cancer progress during treatment. The subject matter disclosed herein addresses these and other needs.

### SUMMARY OF THE DISCLOSURE

In one aspect, disclosed is a lateral flow assay device comprising a first test strip for detecting the presence of CD44, comprising: a. a sample receiving pad for receiving a liquid sample; b. a conjugate test pad in liquid communication with the sample receiving pad and downstream in flow direction from the sample receiving pad, wherein the conjugate test pad comprises a capture reagent deposited thereon, and wherein the capture reagent comprises an anti-CD44 antibody or antigen-binding fragment or derivative thereof with a detectable reporting group; and c. a nitrocellulose membrane in liquid communication with the conjugate pad and downstream in flow direction from the conjugate pad, wherein the nitrocellulose membrane comprises a first detection reagent immobilized thereon, wherein the first detection reagent is capable of binding the capture reagent; wherein CD44 is detected when present in the liquid sample at a level at or around 1.68 ng/mL or higher.

The nitrocellulose membrane can further comprise a second detection reagent immobilized thereon at a position downstream from the first detection reagent, wherein the second detection reagent is an antibody or antibody fragment or derivative capable of binding the capture reagent irrespective of whether the capture reagent is bound to CD44.

The detectable reporting group can be selected from colloidal gold, latex particles, colored dyes, paramagnetic particles, quantum dots, and fluorescent particles. The liquid sample can comprise a bodily fluid, such as saliva or an oral rinse. The anti-CD44 antibody can comprise the BU52 clone antibody. The first detection reagent can be a BU75 clone antibody.

The device can further comprise a test reader to quantitatively determine the amount of CD44 present. The lateral flow assay device can comprise a second test strip for qualitatively detecting total protein, and optionally a third test strip for detecting p16.

Also disclosed is a device for determining whether total protein is above a cutoff level in an oral rinse from a subject, the device comprising a test strip, wherein the test strip changes color when total protein is at or around 0.25 ng/ml or higher. The test strip can change color from yellow to green when total protein is at or around 0.25 ng/ml or higher. The test strip can change color from yellow to green when total protein is at or around 0.30 ng/ml or higher. The test strip can change color from yellow to green when total protein is at or around 0.32 ng/ml or higher.

Further disclosed is a kit comprising the lateral flow assay device disclosed herein, and a sample collection kit. The kit can comprise an oral rinse collection device, such as a cup, and optionally a saline packet comprising 5 mL of saline. The device of the kit can optionally comprise a second and/or a third test strip for detecting total protein and/or p16.

Disclosed herein is a method of detecting a malignant oral disorder in a subject, the method comprising: a. collecting an oral rinse from said subject, b. exposing the lateral flow assay device described herein to the oral rinse, c. detecting a malignant oral disorder in a subject when the first detection reagent binds the capture reagent and a line forms on the device, resulting in a positive test result. The subject with a positive test result can be biopsied based on the result, or referred for further testing to a physician. The oral rinse can also be used to determine if total protein levels from the subject are above a cutoff point or if p16 levels are above a cutoff point.

Further areas of applicability of the disclosure will become apparent from the detailed description provided hereinafter. It should be understood that the detailed description and specific examples are intended for purposes of illustration only and are not intended to limit the scope of the disclosure.

### BRIEF DESCRIPTION OF THE DRAWINGS

The following figures form part of the present specification and are included to further demonstrate certain aspects of the disclosure. The disclosure may be better understood by reference to one or more of these figures in combination with the detailed description of specific embodiments presented herein. The drawings described below are for illustration purposes only. The drawings are not intended to limit the scope of the present teachings in any way.

FIG. 1A-C shows several views of the test strip of a lateral flow assay device. 1A shows a side view. 1B and 1C show top views.

FIG. 2A-E shows several views of the top side of the lateral flow assay device, wherein the lateral flow assay device comprises a test strip enclosed in housing. 2A shows a top view with lateral lines. 2B shows a top view at an angle. 2C shows the top view. 2D shows the end piece. 2E shows a side view.

FIG. 3A-B shows side views of the test strip of the lateral flow assay device. FIG. 3A shows one embodiment. FIG. 3B shows another embodiment.

FIG. 4A-E shows several views of the bottom side of the lateral flow assay device, wherein the lateral flow assay device comprises a test strip enclosed in housing. 4A shows a bottom view with lateral lines. 4B shows a bottom view at an angle. 4C shows the bottom view. 4D shows the end piece. 4E shows a side view.

### DETAILED DESCRIPTION

The disclosure will now be described more fully hereinafter with reference to the accompanying drawings, in which exemplary embodiments of the disclosure are shown. This invention may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the disclosure or of any exemplified term. Likewise, the disclosure is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the disclosure.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the disclosure. As used herein, the singular forms—‘a’′, “an” and “the” are intended to include the plural forms as well, unless the context clearly indicates otherwise. It will be further understood that the terms “comprises” and/or “comprising”, or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top”, may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

As used herein, “around”, “about”, “substantially” or “approximately” shall generally mean within 20 percent, preferably within 10 percent, and more preferably within 5 percent of a given value or range. Numerical quantities given herein are approximate, meaning that the terms “around”, “about”, “substantially” or “approximately” can be inferred if not expressly stated.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

“Optional” or “optionally” means that the subsequently described event or circumstance can or cannot occur, and that the description includes instances where the event or circumstance occurs and instances where it does not.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. The terms “subject” and “patient” are used interchangeably throughout the application.

“Marker” or “biomarker” are used interchangeably herein and refer to a polypeptide (of a particular apparent molecular weight, or, in the case of HA, a molecule made of repeating disaccharide units) which is differentially present in a sample taken from patients having cancer, for example, as compared to a comparable sample taken from control subjects (e.g., a person with a negative diagnosis, normal or healthy subject).

The phrase “differentially present” refers to differences in the quantity and/or the frequency of a marker present in a sample taken from patients having for example, cancer, as compared to a control subject. For example, a marker can be a polypeptide which is present at an elevated level or at a decreased level in samples of patients with head and neck squamous cell carcinoma (HNSCC) compared to samples of control subjects. Alternatively, a marker can be a polypeptide which is detected at a higher frequency or at a lower frequency in samples of patients compared to samples of control subjects. A marker can be differentially present in terms of quantity, frequency or both.

A marker, compound, composition or substance is differentially present between the two samples if the amount of the marker, compound, composition or substance in one sample is statistically significantly different from the amount of the marker, compound, composition or substance in the other sample. For example, a compound is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a marker, compound, composition or substance is differentially present between the two sets of samples if the frequency of detecting the polypeptide in samples of patients is statistically significantly higher or lower than in the control samples. For example, a biomarker is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples. These exemplary values notwithstanding, it is expected that a skilled practitioner can determine cut-off points, etc. that represent a statistically significant difference to determine whether the marker is differentially present.

“Diagnostic” means identifying the presence or nature of a pathologic condition and includes identifying patients who are at risk of developing cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “detection”, “detecting” and the like, can be used in the context of detecting biomarkers, or of detecting cancers like HNSCC (e.g. when positive assay results are obtained). In the latter context, “detecting” and “diagnosing” are considered synonymous.

A “test amount” of a marker refers to an amount of a marker present in a sample being tested. A test amount can be either in absolute amount (e.g., ng/mL) or a relative amount (e.g., relative intensity of signals).

A “diagnostic amount” of a marker refers to an amount of a marker in a subject's sample that is consistent with a diagnosis of cancer or a relative amount of tumor load (e.g., relative intensity of signals).

A “control amount” of a marker can be any amount or a range of amount which is to be compared against a test amount of a marker. For example, a control amount of a marker can be the amount of a marker in a person without cancer. A control amount can be either in absolute amount or a relative amount (e.g., relative intensity of signals).

The terms “polypeptide,” “peptide,” and “protein” are used interchangeably herein to refer to a polymer of amino acid residues, in particular, of naturally-occurring amino acids. The terms apply to amino acid polymers in which one or more amino acid residue is an analog or mimetic of a corresponding naturally-occurring amino acid, as well as to naturally-occurring amino acid polymers. Polypeptides can be modified, e.g., by the addition of carbohydrate residues to form glycoproteins. The terms “polypeptide,” “peptide,” and “protein” include glycoproteins, as well as non-glycoproteins.

“Detectable moiety” or a “label” refers to a composition detectable by spectroscopic, photochemical, biochemical, immunochemical, or chemical means. For example, useful labels include 32p, 35S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains but does not include the heavy chain variable region.

By “binding assay” is meant a biochemical assay wherein the biomarkers are detected by binding to an agent, such as an antibody, through which the detection process is carried out. The detection process can involve radioactive or fluorescent labels, and the like. The assay can involve immobilization of the biomarker, or can take place in solution.

“Immunoassay” is an assay that uses an antibody to specifically bind an antigen (e.g., a marker). The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions can require an antibody that is selected for its specificity for a particular protein. A variety of immunoassay formats can be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

“Sample” as used herein can include polynucleotides, polypeptides, peptides, antibodies fragments and derivatives thereof. A “sample” can be or can come from a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, saliva, blood, skin or hair; fragments and derivatives thereof.

By “at risk of” is meant an increased risk of, compared to a normal subject, or-compared to a control group, e.g. a patient population. Thus a subject “at risk of” developing cancer is at increased risk compared to a normal subject or population, and a subject “at risk of” a recurrence of cancer can be considered at increased risk of having a recurrence as compared to the risk of a recurrence among all treated patients.

“Increased risk” or “elevated risk” mean any statistically significant increase in the probability, e.g., that the subject will develop cancer, or a recurrence thereof. The risk is preferably increased by at least 10%, more preferably at least 20%, and even more preferably at least 50% over the control group with which the comparison is being made.

The term “prognosis” means a prediction about the likely course of disease or disease progression, particularly with respect to likelihood of disease remission, disease relapse, tumor recurrence, metastasis, and death. “Good prognosis” refers to a likelihood that a patient afflicted with cancer, such as head and neck squamous cell carcinoma, will remain disease-free (i.e., cancer-free). “Poor prognosis” refers to a likelihood a patient will have a relapse or recurrence of the underlying cancer or tumor, metastasis, or death. Cancer patients classified as having a “good outcome” remain free of the underlying cancer or tumor. In contrast, “bad outcome” cancer patients experience disease relapse, tumor recurrence, metastasis, or death. In particular examples, the time frame for assessing prognosis and outcome is, for example, less than one year, one, two, three, four, five, six, seven, eight, nine, ten, fifteen, twenty or more years. As used herein, the relevant time for assessing prognosis or disease-free survival time begins with the surgical removal of the tumor or suppression, mitigation, or inhibition of tumor growth. Thus, for example, in particular examples, a “good prognosis” refers to the likelihood that a head and neck squamous cell carcinoma patient will remain free of the underlying cancer or tumor for a period of at least five, more particularly, a period of at least ten years. In further examples, a “bad prognosis” refers to the likelihood that a head and neck squamous cell carcinoma patient will experience disease relapse, tumor recurrence, metastasis, or death within less than five years, more particularly less than ten years. Time frames for assessing prognosis and outcome provided above are illustrative and are not intended to be limiting.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “lifestyle counseling” or “risk factor management counseling” refers to professional counseling administered to a patient regarding the patient's lifestyle. For example, the patient can receive counseling for addictions, such as smoking, drinking, or drug use, or can receive counseling concerning sexual behavior, such as counseling regarding lower-risk sexual behavior, condom use, etc. “Lifestyle counseling” can also refer to diet modification or stress management. Lifestyle counseling is administered by a professional, and can include one or more treatment sessions, literature, professional videos, etc.

The phrase “similar to” as in individuals in the populations have an age similar to that of the subject means that the average age of the population is with 10 year, e.g., within 5 years of the subject. Also, the phrase “similar to” as in individuals in the populations have a race similar to the race of the subject means the subject is or has at least one parent that is the same race as the majority of the individuals in the population. The phrase “similar to” as in individuals in the populations have a history of alcohol consumption, history of tobacco use similar to the subject means the average years of alcohol or smoking are within 10 years, e.g., 5 years, of the subject.

The description will be made as to the embodiments of the disclosure in conjunction with the accompanying drawings. In accordance with the purposes of this invention, as embodied and broadly described herein, this invention, in one aspect, relates to a device for testing a biological sample.

Upper aerodigestive tract (UADT) mucosa progresses through a premalignant phase dysplasia, prior to development of frank malignancy. Identifying lesions in this stage is desirable because dysplasia is reversible (Pindborg, A follow up study of sixty one oral dysplastic precancerous lesions in Indian villagers. Oral Surg Oral Med Oral Pathol (1977) 43:383-90) and can regress spontaneously or with tobacco cessation (Larsson, et al. Reversibility of snuff dippers' lesions in Swedish moist snuff users: a clinical and histologic follow-up study. Oral Pathol Med. (1991) 20(6):258-64; Grizzle, et al. The biology of incipiend, pre-invasive or intraepithelial neoplasia. Cancer Biomark (2010) 9:21-39). Unfortunately, dysplasia is only sometimes visible and often mimics findings that are also seen with benign inflammation. Frequently dysplasia remains occult until further progression resulting in late stage diagnosis (Poh, et al. Direct fluorescence visualization of clinically occult high-risk oral premalignant disease using a simple hand-held device. Head Neck (2007) 29(1):71-6).

CD44, a cell surface transmembrane glycoprotein involved in cell proliferation, cell migration, and tumor initiation (Screaton et al. Proc Natl Acad Sci USA (1992) 89:12160-4; Ponta et al. Nature Rev Mol Cell Biol (2003) 4:33-45; Perez et al. Oral Oncol 2012; Prince et al. Proc Natl Acad Sci USA (2007) 104:973) is overexpressed in premalignant lesions (Hirvikoski et al. Virchows Arch. (1999) 43437-44; Ioachim et al. Histol Histopathol (1999) 14:1113-8). As mucosal changes progress from normal to severe dysplasia, CD44 expression advances from the basal layers to involve all layers of the epithelium (Hirvikoski et al. Virchows Arch. (1999) 43437-44; Ioachim et al. Histol Histopathol (1999) 14:1113-8). Furthermore, CD44 is released by proteinases into a soluble form (solCD44) that is detectable in body fluids (Kajita et al. J. Cell Biol (2001) 153:893-904). Total protein is also an effective tumor marker (Franzmann et al. Head & neck (2012) 34:687-95; Pereira et al. Cancer Biomark (2011)10:241-9; Franzmann et al. Cancer Epidemiol Biomarkers Prev (2007) 16:1348-55).

### Lateral Flow Assay Device

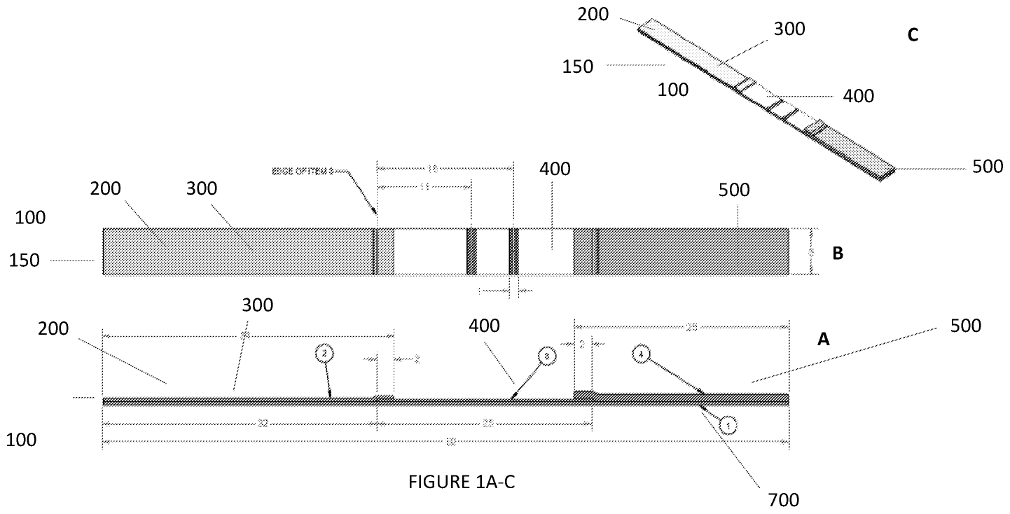

In one embodiment the device includes a lateral flow assay device 100 for qualitatively detecting CD44. As shown in FIG. 1, the lateral flow assay device comprises a first test strip 150 and includes a sample receiving pad 200, a conjugate test pad 300, and a nitrocellulose membrane 400. The test strip device can also comprise an absorbent pad 500. The conjugate test pad 300 is in liquid communication with the sample receiving pad 200 and downstream in flow direction from the sample receiving pad, wherein the conjugate test pad comprises a capture reagent deposited thereon, and wherein the capture reagent can comprise an anti-CD44 antibody or antigen-binding fragment or derivative thereof with a detectable reporting group; and a nitrocellulose membrane 400 in liquid communication with the conjugate pad 300 and downstream in flow direction from the conjugate pad, wherein the nitrocellulose membrane comprises a first detection reagent immobilized thereon, wherein the first detection reagent is capable of binding the capture reagent.

Also disclosed is a lateral flow assay device for determining total protein level. The assay is similar to that shown in FIG. 1, but rather than determine CD44, the total protein level can be determined. One of skill in the art can determine where the cutoff level for detection should be. For example, CD44 can detected when present in the liquid sample at a level at or around 1.00 ng/mL or higher. More specifically, CD44 can be detected when present in the liquid sample at a level at or around 1.10, 1.20, 1.30, 1.40, 1.50, 1.60, 1.70, 1.80, 1.90, or 2.00 ng/mL or higher. Even more specifically, CD44 can be detected at a level of 1.68 ng/mL or higher.

Total protein can be detected when the level is above 0.200 mg/ml. Specifically, total protein can be detected when the level is above 0.210, 0.220, 0.230, 0.240, 0.250, 0.260, 0.270, 0.280, 0.290, 0.300, 0.310, 0.320, 0.330, 0.340, 0.350, 0.360, 0.370, 0.380, 0.390, or 0.400 or higher. Even more specifically, total protein can be detected when the level is 0.325 mg/mL or higher.

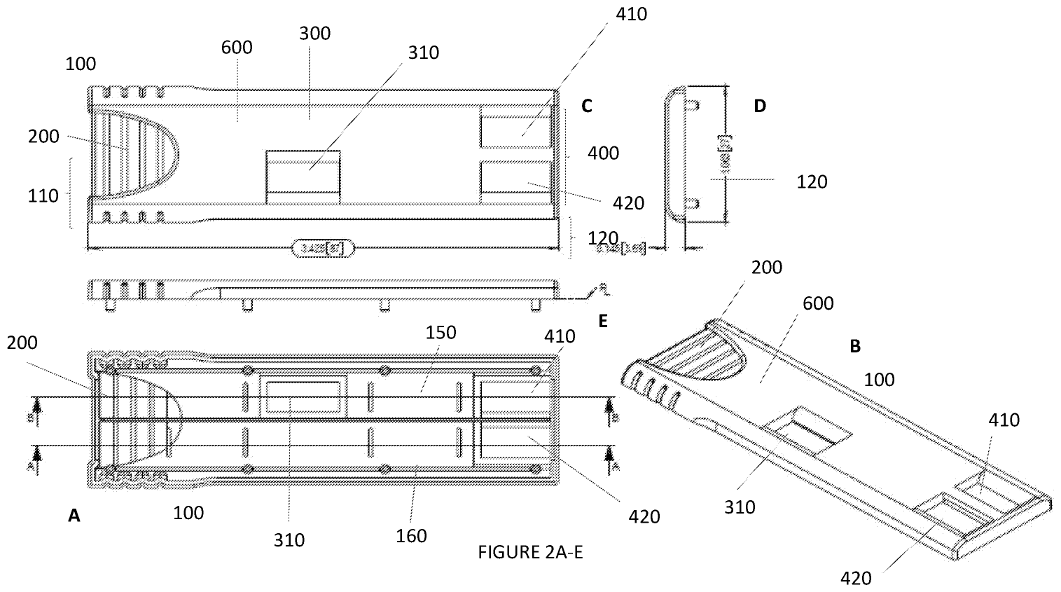

Also disclosed is a lateral flow assay that can detect more than one reagent. For example, the assay can detect CD44 (first test strip 150) and total protein (second test strip 160), or CD44 and p16 (third test strip 170), or CD44, total protein, and p16. When more than one reagent is being tested on the same strip, they can exist in any configuration known to those of skill in the art, such as in tandem (next to each other), on opposite sides back-to-back), or in a circular pattern. FIG. 2 shows a lateral flow assay that can detect more than one reagent simultaneously.

FIGS. 2A and 2B show the top view of a lateral flow assay device 100. The lateral flow assay device has a proximal end 110 and a distal end 120. The proximal end 110 comprises the sample receiving pad 200. The sample receiving pad can be dipped in a liquid sample, such as an oral rinse. As in FIG. 1, the sample receiving pad 200 is in liquid communication with the conjugate test pad 300, which comprises at least a first capture reagent. The conjugate test pad can optionally comprise a second capture reagent, and can optionally comprise a third capture reagent. The capture reagents can act as a control, so that one can determine if the assay device is properly functioning. The results of one of the capture reagents binding the substance of interest, such as CD44, can be viewed in test window 310. A nitrocellulose membrane 400 is in liquid communication with the conjugate test pad 300. The nitrocellulose membrane can comprise at least a first detection reagent, which captures the first capture reagent. The nitrocellulose membrane can also comprise a second detection reagent which captures a second capture reagent 330, and can also optionally comprise a third detection reagent which captures a third capture reagent 340. The results of a first detection reagent binding with its corresponding first capture reagent can be seen in a first test result window 410. The results of a second detection reagent binding with its corresponding second capture reagent can be seen in a second test result window 420. The lateral flow assay device can be encased in housing 600. As can be seen in FIG. 2, the housing can have openings for windows and 310/410/420, as well as an opening at the proximal end for receiving a sample. Test strips are represented by 150/160.

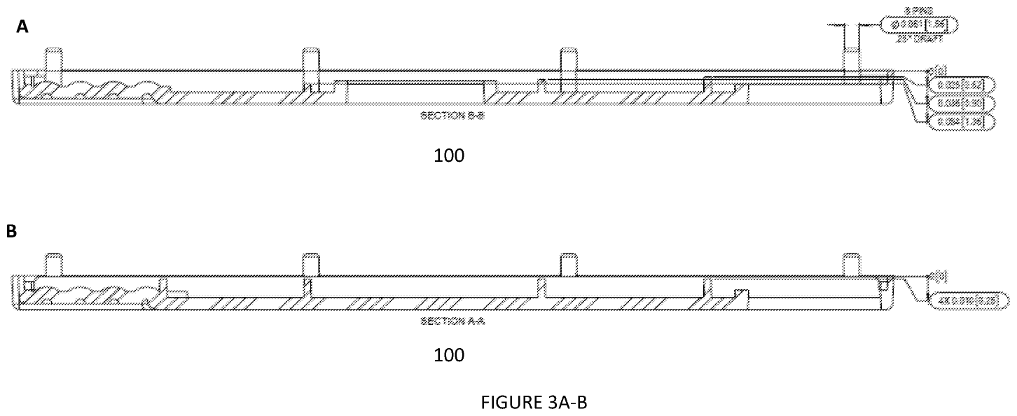

FIG. 3 shows a side, or sectional view of the lateral flow assay device 100.

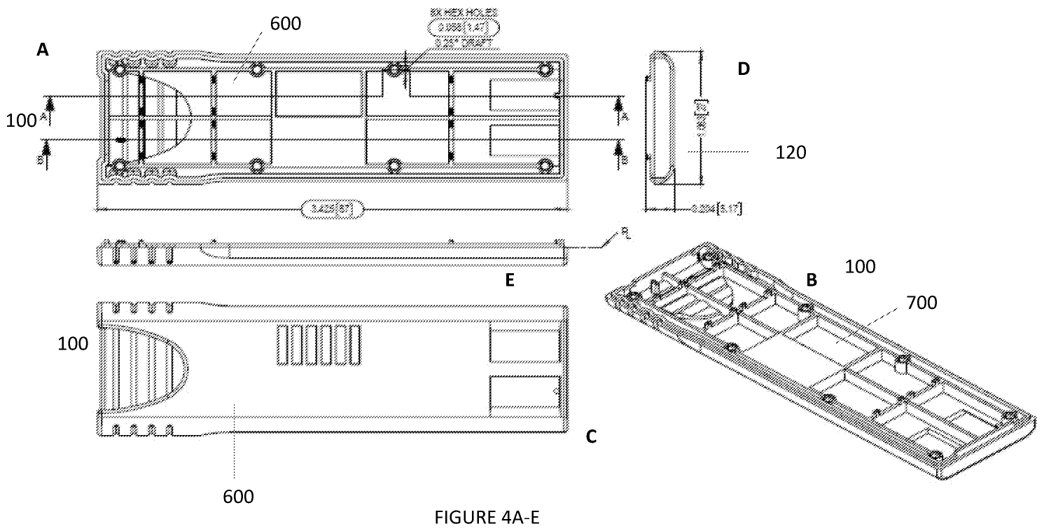

FIG. 4 shows the bottom view of the lateral flow assay device 100 encased in housing 600. The device can comprise backing 700 which is below the test strip 150 and can provide stability and structure to the test strip.

In one embodiment, lateral flow assay device 100 has a length of about 20-300 millimeter (mm), and a width of about 120 mm. In one embodiment, the lateral flow assay device 100 has a length of about 40-160 mm, and a width of about 3-10 mm. In one embodiment, the lateral flow assay device 100 has a length of about 80 mm, and a width of about 4.7 mm.

The lateral flow assay device 100 has a proximal end 110 and an opposite, distal end 120. In one embodiment, the sample receiving pad 200 is disposed on the proximal end 110 of the device 100 and configured to receive the biological sample. The sample receiving pad 200 and the conjugate test pad 300 can be contiguous or overlapping. The conjugate test pad can be treated with surfactants, proteins, etc. In one embodiment, the conjugate test pad 300 includes colored latex or anything that can be visually or electronically detected that serves as labeled material. The labeled material typically is applied with stabilizing agents such as sucrose, surfactants, buffers, proteins, and sometimes salts. This is called a conjugate diluent and is important to make the test work properly with the sample being tested. The conjugate test pad can be an Ahlstrom 8951 Conjugate Pad (34 mm).

The nitrocellulose membrane 400 can be in fluid communication with the conjugate test pad 300. The nitrocellulose membrane can be a Sartorius CN95 nitrocellulose membrane, and can be about 25 mm. The nitrocellulose membrane can comprise a BU75 clone capture/test line antibody. The control line can include goat anti-mouse (GAM) antibody, so as to indicate the amount of CD44 in the biological sample. In one embodiment, a color of each of the control line and the test line is compared to a color intensity chart to evaluate the feature of a protein of interest, such as the concentration of the protein of interest, where the color intensity chart shows a relationship between a color intensity and the concentration of a protein of interest.

In one embodiment, a color of each of the control line and the test line is evaluated by a reader. In the assays disclosed herein, a detectable signal that includes at least one of emission (e.g., fluorescence), color, reflectance, diffuse scattering (i.e., scattering and absorbance), elastic light scattering, chemiluminescence, chemifluorescence, transmission, or absorbance can be used. A lens and a detector (e.g., a digital camera device) can be used to collect data from the assay.

The reader can be a lateral flow reader, such as an Axxin Lateral Flow Reader. Electronic reflectance can be measured by a reflectance reader. Another example is the SNAP® Reader analyzer, manufactured and distributed by IDEXX Laboratories, Inc., an image-analysis instrument which includes a digital camera. The analyzer can take a digital picture as test results are developing, and the software of the analyzer uses algorithms specific to the test to calculate the test results from these digital images. Other analyzers exist for reading lateral flow devices based on colloidal gold technology, such as: DCN Technologies, Carlsbad, Calif.; and the ESEQuant™ lateral flow reader from Qiegen Nev., Venlow, Netherlands.

As described above, a color of each of the control line and the test line can be evaluated by taking a picture of the corresponding line and analyzing the picture taken. In one embodiment, the concentration of the protein of interest is determined using an electronic reader with a CCD camera, a reflectance reader, or other optical methods to give quantitative results.

The digital camera device for use with the reader can be a camera phone. In other embodiments, the digital camera device can be essentially any camera phone or digital camera. For example, the digital camera device can be a camera phone or digital camera that has an onboard image processing capability and the ability to communicate wirelessly with a database. In one embodiment, a smart phone can be used with the digital camera, and the smart phone can comprise an app for reading the test results.

In one embodiment, the conjugate test pad 300 has a length of about 5-100 mm, and a width of about 1-20 mm. In one embodiment, the test pad 300 has a length of about 12-50 mm, and a width of about 3-10 mm. In one embodiment, the test pad 300 has a length of about 25 mm, and a width of about 4.7 mm. The test pad 300 can be disposed on the first test strip 150 such that the conjugate test pad 300 has a portion in contact with the sample receiving pad 200 and configured to test the biological sample 210 received from the sample receiving pad 200. In one embodiment, one end portion of the sample receiving pad 200 is disposed over one end portion of the conjugate test pad 300 and is in contact with the test pad 300. In certain embodiments, the sample receiving pad 200 overlaps the test pad 300 by about 1-6 mm. In one embodiment, the overlap is about 2-3 mm. In one embodiment, the test strip(s) 150/160/170 are formed of plastic, paper, metal, glass, or the like. In one embodiment, the substrate has single-sided adhesive. In one embodiment, the substrate has double-sided adhesive.

In one embodiment, the test strip(s) 150/160/170 can be flexible.

In one embodiment, the first test strip 150 includes a CD44 strip configured to evaluate an amount of CD44 proteins in the biological sample. CD44 molecule is a cell surface glycoprotein involved in cell-cell interaction, cell adhesion and migration. In humans, the CD44 antigen is encoded by the CD44 gene on chromosome 11. In certain embodiments, the CD44 is directed to human CD44 protein.

In a second embodiment, the second test strip 160 includes a total protein strip configured to evaluate the amount of total protein in the biological sample.

In a third embodiment, the third test strip 170 includes a p16 test strip to evaluate the amount of p16 in a biological sample. Expression of p16^{INK4A} (p16 positive) is highly correlated with human papilloma virus (HPV) infection in head and neck squamous cell carcinoma (HNSCC).

In one embodiment, the conjugate test pad 300 contains at least one of BU52 clone antibody conjugated to 40 nm colloidal gold particles, and conjugate diluent and conjugate pad blocking buffer.

In one embodiment, the lateral flow assay device 100 further includes an absorbent pad 500 disposed on the distal surface 120 of the device. In one embodiment, the absorbent pad 500 has a length of about 5-100 mm, and a width of about 1-20 mm. In one embodiment, the absorbent pad 500 has a length of about 12-50 mm, and a width of about 3-10 mm. In one embodiment, the absorbent pad 500 has a length of about 25 mm, and a width of about 4.7 mm. In one embodiment, one end of the absorbent pad 500 is in contact with the nitrocellulose membrane 400. In one embodiment, the absorbent pad is an Ahlstrom 222 absorbent sink pad.

In one embodiment, the lateral flow assay device comprises housing 600. Housing 600 can encase the test strips 150/160/170, and can have a length of about 15-250 mm, and a width of about 1-20 mm. In one embodiment, the housing 600 has a length of about 30-120 mm, and a width of about 3-10 mm. In one embodiment, the housing 600 has a length of about 65 mm, and a width of about 4.7 mm. The housing 600 can cover all or part of the sample receiving pad 200, the conjugate test pad 300, the nitrocellulose membrane 400, and the absorbent pad 500. In one embodiment, the part of the sample receiving pad 200 exposed out of the housing 600 has a length of about 2-40 mm. In one embodiment, the part of the sample receiving pad 200 exposed out of the housing 600 has a length of about 5-20 mm. In one embodiment, the part of the sample receiving pad 200 exposed out of the housing 600 has a length of about 10 mm. In one embodiment, the part of the absorbent pad 500 exposed out of the housing 600 has a length of about 0.5-10 mm. In one embodiment, the part of the absorbent pad 500 exposed out of the housing 600 has a length of about 1-5 mm. In one embodiment, the part of the absorbent pad 500 exposed out of the housing 600 has a length of about 2 mm. The housing 600 can have one or two transparent arrows corresponding to the area of the sample receiving pad 200. The transparent arrows can be used to indicate the direction of the test strip 150/160/170 in the container and may be used to aid the observation or evaluation of the test result of the assay. In one embodiment, the end of the strip having the sample receiving pad 200 is disposed at the open end of an end-sealed container, and the arrows on the housing 600 are directed to the open end of the container. In one embodiment, a portion of the housing 600 covering the conjugate test pad 300 and the nitrocellulose membrane 400 is transparent, such that the control line and the test line are exposed for observation.

The lateral flow assay device can also comprise a backing card 700. The backing card can run the length of the test strips 150/160/170, and can be a Kenosha Vinyl Backing Card 80 mm×300 mm.

In one embodiment, the device further includes a container configured to accommodate the biological sample.

In one embodiment, when in operation, the proximal end 110 of the lateral flow assay device 100 comprising the sample receiving pad 200 is placed into the container for receiving the biological sample therein.

In one embodiment, the device is reusable or disposable.

In one embodiment, the second test strip 160 is a total protein (TP) pad configured to test total protein amount in the sample from the second test strip 160. In one embodiment, the TP test strip 160 is a Teco Diagnostics TP pad or equivalent. In one embodiment, the color of the TP test strip 160 is an indicative of the amount of total protein. An unused test device will have no visible lines with a yellow color total protein pad. A positive test will either have the CD44 test line and control line present, or the green total protein pad, while a negative result will have no CD44 test line present, but a visual control line present and a yellow or yellow/green color total protein pad.

In one example, a device includes a liquid sample container, housing 600, and multiple test strips 150/160/170 within the housing. The container has a sealed end and an open end. The housing 600 and the multiple test strips 150/160/170 are received in the container. When in operation, the housing 600 with the multiple test strips 150/160/170 is pulled out from the container. A biological sample, such as 5 milliliters of saline of an object, can be added to the container. Then the housing with multiple test strips are insert back into the container such that the sample receiving pads 200 of the multiple test strips 150/160/170 are immersed in the biological sample. The housing 600 with the multiple test strips 150/160/170 are kept in the biological sample for a period of time, and the testing results can be evaluated for each test strip.

In another example, the device of the disclosure can be used for early detection or diagnosis of cancer or cancer risks, where the first test strip 150 is configured to detect quantity of CD44 protein and the second test strip 160 is configured to detect quantity of total protein. Optionally, a third test strip 170 can be used to detect p16. The result can be used to early detect or evaluate risks of head and neck squamous cell carcinoma (HNSCC). In one embodiment, the result can be used to detect the risk of cancer occurrence. In one embodiment, the result can be used to evaluate success of a cancer treatment. In one embodiment, result can be used to predict the recurrence of a cancer after successful treatment of the cancer.

In a further example, the disclosure is directed to a kit for early detection of cancer risks. The kit includes a first test strip 150 for detection of CD44 protein and a second test strip 160 for detection of total protein. The kit can further optionally include a third test strip 170 for detecting p16 levels. The kit further includes a straw with a scaled end and an open end, for receiving the test strip(s), and for receiving biological samples to be tested.

The liquid sample container 210 can comprise a transparent cup body, a cap and a support member contained in the cup body. The cup body has a side wall and a bottom wall. The side wall is in a tubular shape, and defined by an upper diameter corresponding to the cap and a lower diameter corresponding to the circumference of the bottom wall. The upper diameter may be equal to or greater than the lower diameter.

The housing 600 can be flexible and include multiple notched receptacles connected side by side, such that the support member has a rectangular plate form. Each notched receptacle has a sealed end and an open end. Each notched receptacle can include one test strip 150/160/170. In certain embodiments, the sealed end of each notched receptacle is positioned towards the top of the cup body.

The cup body can have instructions for explanation of the testing result. In one embodiment, a positive or a negative result corresponds to two colored lines revealed on a corresponding test strip, where the C line indicates the control line and the T line indicates the test line. In one embodiment, a negative or a positive result corresponds to one colored C line revealed on a corresponding test strip, where T line cannot be seen. Whether two colored lines or a one colored line corresponds to a positive result or a negative result depends on the chemistry used in the testing.

In one implementation, the biological sample may be a saliva sample taken after swishing around an oral solution in the mouth, referred to herein as an oral rinse. The container can comprise a packet of 5 mL of sterile saline for swishing in the mouth. The patient should be instructed to swish the entire 5 mL of sterile saline in their mouth, being sure to swish through the teeth, tongue, and cheeks for 5 seconds, then gargle for 5 seconds and expectorate the entire solution into the sample cup. The device can then be immersed into the container at a 90 degree angle for at least 3 seconds, then removed. The results can be read after 10 minutes.

Disclosed herein is a method of detecting a malignant oral disorder in a subject, the method comprising: a) collecting an oral rinse from said subject, b) exposing the lateral flow assay device described herein to the oral rinse, c. detecting a malignant oral disorder in a subject when the first detection reagent binds the capture reagent and a line forms on the device, resulting in a positive test result. The subject with a positive test result can be biopsied based on the result, or referred for further testing to a physician. The oral rinse can also used to determine if total protein levels from the subject are above a cutoff point.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the disclosure pertains without departing from its spirit and scope. Accordingly, the scope of the disclosure is defined by the appended claims as well as the disclosure including drawings.

1. A lateral flow assay device comprising a first test strip for detecting the presence of CD44, comprising:
a. a sample receiving pad for receiving a liquid sample;
b. a conjugate test pad in liquid communication with the sample receiving pad and downstream in flow direction from the sample receiving pad, wherein the conjugate test pad comprises a capture reagent deposited thereon, and wherein the capture reagent comprises an anti-CD44 antibody or antigen-binding fragment or derivative thereof with a detectable reporting group; and
c. a nitrocellulose membrane in liquid communication with the conjugate pad and downstream in flow direction from the conjugate pad, wherein the nitrocellulose membrane comprises a first detection reagent immobilized thereon, wherein the first detection reagent is capable of binding the capture reagent;
wherein CD44 is detected when present in the liquid sample at a level at or around 1.68 ng/mL or higher.

2. The device of claim 1, wherein the nitrocellulose membrane further comprises a second detection reagent immobilized thereon at a position downstream from the first detection reagent, wherein the second detection reagent is an antibody or antibody fragment or derivative capable of binding the capture reagent irrespective of whether the capture reagent is bound to CD44.

3. The device of claim 1, wherein the detectable reporting group is selected from colloidal gold, latex particles, colored dyes, paramagnetic particles, quantum dots, and fluorescent particles.

4. The device of claim 1, wherein the liquid sample comprises a bodily fluid.

5. The device of claim 4, wherein the bodily fluid is saliva.

6. The device of claim 1, wherein the liquid sample comprises an oral rinse.

7. The device of claim 1, wherein the anti-CD44 antibody comprises BU52 clone antibody.

8. The device of claim 7, wherein the first detection reagent is BU75 clone antibody.

9. The device of claim 1, wherein the device further comprises a test reader to quantitatively determine the amount of CD44 present.

10. A device for determining whether total protein is above a cutoff level in an oral rinse from a subject, the device comprising a test strip, wherein the test strip changes color when total protein is at or around 0.25 ng/ml or higher.

11. The device of claim 10, wherein the test strip changes color from yellow to green when total protein is at or around 0.25 ng/ml or higher.

12. The device of claim 11, wherein the test strip changes color from yellow to green when total protein is at or around 0.30 ng/ml or higher.

13. The device of claim 12, wherein the test strip changes color from yellow to green when total protein is at or around 0.32 ng/ml or higher.

14. The device of claim 1, wherein the lateral flow assay device comprises a second test strip for qualitatively detecting total protein.

15. The device of claim 1 or 14, wherein the lateral flow assay device comprises a third test strip for detecting p16.

16. A kit comprising the device of claim 1 and a sample collection kit.

17. The kit of claim 16, wherein the sample collection kit comprises an oral rinse kit.

18. The kit of claim 17, wherein the oral rinse kit comprises a saline packet and a sample collection cup.

19. The kit of claim 18, wherein the saline packet comprises 5 mL of saline.

20. The kit of claim 17, wherein the device of the kit further comprises a second test strip for determining whether total protein is above a cutoff point.

21-29. (canceled)
